(19) United States
(12) Patent Application Publication
(10) Pub. No.: US 2010/0047312 A1
(43) Pub. Date: Feb. 25, 2010

(54) USE OF BIOACTIVE AND RADIOPAQUE MATERIAL FOR STENT COATING

(76) Inventor: Eric Wittchow, Nuernberg (DE)

Correspondence Address: GREER, BURNS & CRAIN, 300 S WACKER DR, 25TH FLOOR, CHICAGO, IL, 60606, US

(21) Appl. No.: 12/540,889

(22) Filed: Aug. 13, 2009

(30) Foreign Application Priority Data
Aug. 19, 2008 (DE) 10 2008 038 368.6

(51) Int. Cl. A61K 9/00; A61F 2/06; A61K 31/28; A61K 31/7004; A61K 31/66; A61P 9/10

(52) U.S. Cl. 424/423; 623/1.46; 514/495; 514/23; 514/129

(57) ABSTRACT

The invention relates to a stent having a coating or cavity filling comprising or containing an organic Au complex.

### FIELD OF THE INVENTION

The invention relates to a stent coated with a bioactive and also radiopaque material and use thereof for production of such stents.

### BACKGROUND OF THE INVENTION

Implantation of stents has become established as one of the most effective therapeutic measures for treatment of vascular diseases. The purpose of stents is to assume a supporting function in the hollow organs of a patient. Stents of a traditional design therefore have a filigree supporting structure comprising metallic struts, which are initially present in a compressed form for introduction into the body and are widened at the site of application. One of the main areas of application of such stents is for permanent or temporary dilatation of vascular stenoses, in particular stenoses of the coronary vessels, and maintaining their patency. In addition, aneurysm stents, which serve to support damaged vascular walls, are also known.

Stents have a circumferential wall with a sufficient supporting force to keep the stenosed vessel open to the desired extent and they have a tubular base body through which the blood flow passes unhindered. The circumferential wall is usually formed by a mesh-like supporting structure which makes it possible to insert the stent in a compressed state with a small outside diameter up to the stenosis in the respective vessel to be treated and to dilate the vessel there, e.g., with the help of a balloon catheter to the extent that the vessel has the desired enlarged inside diameter. The stent positioning and expansion operation during the procedure and the subsequent position of the stent in the tissue after the end of the procedure must be monitored by a cardiologist. This can be accomplished by imaging methods, e.g., by radiology.

The stent has a base body of an implant material. An implant material is a nonviable material that is used for an application in medicine and enters into an interaction with biological systems. Biocompatibility is a basic prerequisite for use of a material as an implant material which comes in contact with the body's environment when used as intended. Biocompatibility is understood to be the ability of a material to induce an appropriate tissue reaction in a specific application. This includes an adaptation of the chemical, physical, biological and morphological surface properties of an implant to the recipient tissue with the goal of a clinically desired interaction. The biocompatibility of an implant material also depends on the chronological course of the reaction of the biosystem into which it is implanted. Thus relatively short-term irritation and inflammation occur and can lead to tissue changes. Biological systems thus react in various ways, depending on the properties of the implant material. Implant materials can be subdivided into bioactive, bioinert and degradable/absorbable materials, according to the reaction of the biosystem.

Implant materials for stents comprise polymers, metallic materials and ceramic materials (e.g., as the coating). Biocompatible metals and metal alloys for permanent implants contain, for example, stainless steels (e.g., 316L), cobalt base alloys (e.g., CoCrMo casting alloys, CoCrMo forged alloys, CoCrWNi forged alloys and CoCrNiMo forged alloys, L605), pure titanium and titanium alloys (e.g., cp titanium, TiAl6V4 or TiAl6Nb7) and gold alloys. In the field of biocorrodible stents, the use of magnesium or pure iron and biocorrodible basic alloys of the elements magnesium, iron, zinc, molybdenum and tungsten is proposed.

A biological reaction to polymeric, ceramic or metallic implant materials depends on the concentration, duration of action and how administered. The presence of an implant material often leads to inflammation reactions, where the triggering factors may be mechanical stimuli, chemical substances or metabolic products. The inflammation process is usually accompanied by migration of neutrophilic granulocytes and monocytes through the vascular walls, migration of lymphocyte effecter cells, forming antibodies to the activation of the complement system with the release of complement factors, which act as mediators, and ultimately the activation of blood coagulation. An immunological response is usually closely associated with the inflammation reaction and may lead to sensitization and allergization. Known metal allergens include, for example, nickel, chromium and cobalt, which are also used as alloy components in many surgical implants. An important problem with stent implantation in blood vessels is in-stent restenosis due to excessive neointimal growth, which is caused by a marked proliferation of arterial smooth muscle cells and a chronic inflammation reaction.

It is known that a greater biocompatibility and thus an improved restenosis rate can be achieved when implant materials are provided with coatings of materials that are especially biocompatible. These materials are usually of an organic or synthetic polymer type and are partially of natural origin. Additional strategies for preventing restenosis are concentrated on inhibiting proliferation through medication, e.g., treatment with cytostatics. For example, the active ingredients can be supplied in the form of a coating on the implant surface.

### SUMMARY OF THE INVENTION

Despite the progress achieved, there is still a high demand for achieving better integration of a stent into its biological environment and thereby reducing the rate of restenosis while at the same time ensuring adequate imageability of the stent during and after application.

This object is achieved by providing a stent having a coating or cavity filling consisting of or containing an organic gold [Au] complex.

The present invention is based on the finding that gold in the form of complexes, in particular gold ions, Au(I) and Au(III), as well as radiopaque markers, acts as a radiopaque marker for X-ray imaging and also as a restenosis-inhibiting and/or restenosis-preventing substance. Two essential properties of a coated stent, namely imageability of the stent in tissue and restenosis prevention, are thus combined in a single substance. Consequently, the production and coating methods can be greatly simplified. First, the number of steps required to produce the stent can be reduced because production of the marker and production of the active coating/cavity filling are combined. Secondly, there is a simplification in comparison with traditional coatings/cavity fillings, which also contain the marker as a biologically active substance. By combining these components, it is possible to avoid the incompatibility of the marker and active substance, e.g., different solubilities, and to facilitate optimization of the system with regard to the desired application.

According to a first variant, the base body of the stent thus has a coating containing or consisting of the inventive Au complex. A coating in the sense of the invention is an application of the components of the coating to the base body of the stent in at least some sections. The entire surface of the base body of the stent is preferably covered by the coating. The layer thickness is preferably in the range of 1 nm to 100 μm, especially preferably 300 nm to 15 μm. The amount by weight of the components forming the coating in the Au complex or the Au complex in the coating is preferably at least 40%, especially preferably at least 70%. The coating may be applied directly to the implant surface. The processing may be performed by standard coating methods. Single-layer systems as well as multilayer systems (for example so-called base coat, drug coat or top coat layers) may be created. The coating may be applied directly to the base body of the stent or additional layers may be provided in between, serving to promote adhesion, for example.

Alternatively, the Au complex may be in the form of a cavity filling or as a component of a cavity filling. The stent therefore has one or more cavities. Cavities occur on the surface of the stent, for example, and can be created by laser ablation in micrometer dimensions. In the case of stents having a biodegradable base body, a cavity may also be provided on the interior of the base body, so that the material is released only after it has been exposed. With this embodiment of the cavity, those skilled in the art may orient themselves on the systems described in the prior art.

In addition to the use of the inventive gold complexes, the coating or cavity filling may contain other ingredients, in particular an organic polymer matrix in which the Au complex is embedded in a finely dispersed form. In other words, the stent has a coating or cavity filling consisting of a polymer carrier matrix with embedded Au complex or containing these components. The carrier matrix may contain other pharmaceutical ingredients, other X-ray markers or magnetic resonance markers in particular.

Because of its high atomic weight of 197, gold is a suitable X-ray marker and has also been widely used as such.

According to a preferred embodiment of the invention, the Au complex is an anticancer active ingredient or immunomodulating active ingredient. Au complexes such as Tauredon® are therefore used to treat inflammatory diseases such as rheumatoid arthritis. Such Au complexes thus have immunomodulating effects. Au complexes have recently also been described as being suitable for use in tumor therapy (I. Kostova, Anti-Cancer Agents in Medicinal Chemistry, 2006, 6, 19-32). Such Au complexes thus have anticancer effects. Active ingredients with anticancer or immunomodulating effects are currently of special interest as agents for preventing a restenosis. The active ingredient is especially preferably selected from the group comprising aurothiomalate, aurothioglucose, bis(thiosulfate) Au(I), thiopropane sulfate-S—Au(I), thiopropanol sulfonate-S—Au(I), 1,2-bis(diphenylphosphine)ethane Au(I), 1,2-bis(dipyridylphosphino)ethane Au(I) and tetracis((tris(hydroxymethyl))phosphine) Au(I). Especially preferred compounds also include those in which the Au(I) is complexed by a phosphorus atom as well as by a sulfur atom so the complex has the structural unit P—Au—S. One example of this class of compounds is tetraacetylthioglucose-Au(I)-triethylphosphine, which is known under the name Auranofin.

Inventive Au complexes are preferably complexes of gold ions such as Au(I) and Au(III). In preferred Au complexes, the gold ion forms a stable complex with at least one or more elements of main groups V. and VI. of the Periodic System (IUPAC groups 15 and 16), whereby in one complex, different elements may be involved in the complexing (heterogeneous complex).

If the Au complex is an Au(I) complex, then it preferably has one or more sulfur ligands. This would be in particular the thiolate ligand, the thiosulfate ligand, the disulfide ligand and the thiocarbohydrate ligand (e.g., thioglucose).

Also preferred are Au(I) complexes having one or more tertiary phosphine ligands. In particular the phosphine ligand is selected from the group comprising triethylphosphine, 1,2-bis(diphenylphosphine)ethane, 1,2-bis(dipyridylphosphine)ethane, and tris(hydroxy-methyl)phosphine.

If the Au complex is an Au(III) complex, then it preferably has one or more multidentate ligands containing nitrogen. The N-containing ligand is selected in particular from the group comprising ethylenediamine, diethylenediamine, N-benzyl-N,N-dimethylamine; from bispyridyl ligands, in particular (bispyridyl)(OH)₂, (6-(1,1-dimethylbenzyl)-2,2′-bipyridine-H)(OH); or from trichloro(2-pyridylmethanol), dichloro(N-ethylsalicylaldiminate), trichloro-(diethylenediamine), and trichloro(bisethylenediamine). Especially preferred are dithio-carbamate ligands.

Additional Au complexes suitable for the purposes of the invention are described in the literature and means of synthesis of the inventive Au complexes are known to those skilled in the art. As examples, reference can be made to some relevant publications in which anticancer properties of these compounds are discussed:
Kostova, Anti-Cancer Agents in Medicinal Chemistry, 2006, 6, 19-32.
V. Milacic, Histol. Histopathol. 2008, 23, 101-108.
S. L. Best, P. J. Sadler, Gold Bulletin, 1996, 29, 87.
S. P. Fricker, Gold Bulletin, 1996, 29, 53.
R. V. Parish, S. M. Cottrill, Gold Bulletin 1987, 20, 3.
E. R. T. Tiekink, Gold Bulletin 2003, 36, 117.
M. A. Mazid et al., J. Chem. Soc. Chem. Commun., 1980, 1261.
R. C. Elder et al., J. Am. Chem. Soc., 1985, 107, 5024.
M. J. McKeage et al., J. Coord. Chem. Rev., 2002, 232, 127.
M. J. McKeage et al., Cancer Chemother. Pharmacol., 2000, 46, 343.
F. Caruso et al., J. Med. Chem., 2003, 46, 1737.
N. Pillarsetty et al., J. Med. Chem., 2003, 46, 1130.
R. G. Buckley et al., 1996, 39, 5208.
M. Coronello et al., Oncol. Res., 2000, 12, 361.
B. Bruni et al., Croatica Chemica Acta, 1999, 72, 221.

Those skilled in the art can obtain preliminary information about the synthesis methods to be used in synthesis of the Au complexes from these publications.

If the stent is made entirely or in part of a biocorrodible metallic material, in particular a magnesium alloy, then there are special demands of the marker. Elemental gold is not suitable for these materials because it tends to form local elements with the surrounding base metals such as magnesium, thus greatly accelerating the dissolution of the less noble metal. Due to the strong complexing in organic gold complexes, the risk of a reduction in the gold ion to elemental gold and formation of local elements is reduced or at best eliminated completely, so the use of the Au complexes with stents made of a biocorrodible metallic material is especially preferred. The metallic base body preferably consists of magnesium, a biocorrodible magnesium alloy, pure iron, a biocorrodible iron alloy, a biocorrodible tungsten alloy, a biocorrodible zinc alloy or a biocorrodible molybdenum alloy. The biocorrodible metallic material is in particular a magnesium alloy.

Alloys and elements in which degradation/rearrangement takes place in a physiological environment so that the part of the implant made of the material is no longer present at all or at least predominately are understood to be biocorrodible in the sense of the invention.

Magnesium alloy, iron alloy, zinc alloy, molybdenum alloy or tungsten alloy is understood to be mainly a metallic structure in which the main component is magnesium, iron, zinc, molybdenum or tungsten. The main component is the alloy component present in the largest amount by weight in the alloy. The main component preferably amounts to more than 50 wt %, in particular more than 70 wt %. The composition of the alloy is to be selected so that it is biocorrodible. Artificial plasma such as that stipulated according EN ISO 10993-15:2000 for biocorrosion studies is used as the test medium for testing the corrosion behavior of an alloy in question (NaCl 6.8 g/L, CaCl₂ 0.2 g/L, KCl 0.4 g/L, MgSO₄ 0.1 g/L, NaHCO₃ 2.2 g/L, Na₂HPO₄ 0.126 g/L, NaH₂PO₄ 0.026 g/L). A sample of the alloy to be tested is therefore stored at 37° C. in a sealed sample container with a defined amount of the test medium. At intervals of a few hours up to several months, based on the corrosion behavior to be expected, the samples are removed and tested for traces of corrosion in the known way. The artificial plasma according to EN ISO 10993-15:2000 corresponds to a medium resembling blood and thus constitutes a possibility of reproducibly simulating a physiological environment in the sense of the invention.

The coating of the inventive stents may contain one or more different Au(I) or Au(III) complexes or may optionally consists of such a mixture.

The invention also relates to the use of organic Au complexes for coating or filling the cavity of stents.

The invention is explained in greater detail below on the basis of an exemplary embodiment.

### DETAILED DESCRIPTION OF THE INVENTION

### Exemplary Embodiment 1—Coating of a Stent

A stent made of the biocorrodible magnesium alloy WE43 (97 wt % magnesium, 4 wt % yttrium, 3 wt % rare earth metals not including yttrium) is coated as follows:

A solution of Auranofin (M=679.5 g/mol) in THF (10 wt %) is prepared at RT. This solution is mixed with a second solution of polylactide (L210; Boehringer-Ingelheim) in THF (10 wt %) at RT, such that the gold salt and the polylactide are in a weight ratio of 1:1.

The stent is cleaned to remove dust and residues and is clamped in a suitable stent coating apparatus (DES coater, in-house development of Biotronik). With the help of an airbrush system (EFD or spraying system) the rotating stent is coated with the gold salt/polymer mixture on a half side under constant ambient conditions (room temperature; 42% atmospheric humidity). At a nozzle distance of 20 mm, an 18-mm-long stent is coated after approximately 10 minutes. After reaching the intended layer weight, the stent is dried for 5 minutes at room temperature before the uncoated side is coated in the same way after rotating the stent and clamping it again. The completely coated stent is dried for 36 hours at 40° C. in a vacuum oven (Vakucell; MMM).

The layer thickness of the applied coating is approximately 3-5 μm.

It will be apparent to those skilled in the art that numerous modifications and variations of the described examples and embodiments are possible in light of the above teaching. The disclosed examples and embodiments are presented for purposes of illustration only. Therefore, it is the intent to cover all such modifications and alternate embodiments as may come within the true scope of this invention.

1. A stent having one or more of coating or cavity filling comprising an organic Au complex.

2. The stent according to claim 1, wherein the stent comprises a biocorrodible metallic material.

3. The stent according to claim 2, wherein the biocorrodible metallic material is a magnesium alloy.

4. The stent according to claim 1, wherein the Au complex is one or more of an Au(I) and an Au(III) complex.

5. The stent according to claim 1, wherein the Au complex is an Au(I) complex and has one or more tertiary phosphine ligands.

6. The stent according to claim 5, wherein the phosphine ligand is selected from the group comprising 1,2-bis(diphenylphosphine)ethane, 1,2-bis(dipyridylphosphine)-ethane, and tris(hydroxymethyl) phosphine.

7. The stent according to claim 1, wherein the Au complex is an Au(III) complex and has one or more multidentate N-containing ligands.

8. The stent according to claim 7, wherein the N-containing ligand is selected from the group comprising ethylenediamine, diethylenediamine, N-benzyl-N,N-dimethylamine; bispyridyl ligands, (bispyridyl)(OH)₂, (6-(1,1-dimethylbenzyl)-2,2′-bipyridine-H)(OH); trichloro(2-pyridylmethanol), dichloro-(N-ethylsalicylaldiminate), trichloro(diethylenediamine), trichloro(bisethylene-diamine) and dithiocarbamate.

9. The stent according to claim 1, wherein the Au complex is one or more of an anticancer and immunomodulating active ingredient.

10. The stent according to claim 9, wherein the active ingredient is selected from the group comprising aurothiomalate, aurothioglucose, Auranofin, bis(thiosulfate) Au(I), thiopropanesulfate-S—Au(I), tetraacetyl-P-D thioglucose Au(I) triethyl-phosphine, 1,2-bis(diphenylphosphine)ethane Au(I), 1,2-bis(dipyridylphosphino)-ethane Au(I), tetrakis((tris(hydroxymethyl))phosphine) Au(I) or dithiocarbamate Au(III).

11. The stent according to claim 1, wherein the Au complex is embedded in an organic polymer matrix which is applied as one or more of a coating or cavity filling to the stent.

12. (canceled)

13. The stent according to claim 1 wherein the one or more of a coating or cavity filling consists essentially of the organic Au complex.

14. The stent according to claim 2 wherein the stent consists essentially of the biocorrodible material.

15. A method for making stents comprising the steps of applying one or more of a coating and a cavity filling that comprises an Au complex to the stent.

16. A stent comprising a biocorrodible magnesium alloy material and having one or more of a coating or cavity filling comprising an organic Au complex embedded in a polymer matrix, the Au complex being one or more of an Au(I) complex with at least one tertiary phosphine ligand and a Au(III) complex having at least one multidentate N-containing ligands.
